(19) United States
(12) Patent Application Publication
(10) Pub. No.: US 2005/0137656 A1
(43) Pub. Date: Jun. 23, 2005

(54) Acoustic-optical therapeutical devices and methods

(75) Inventor: Henryk Malak, Ellicott City, MD (US)

Correspondence Address: Henryk Malak, 8444 High Ridge Road, Ellicott City, MD, 21043, US

(73) Assignee: American Environmental Systems, Inc.

(21) Appl. No.: 10/846,886

(22) Filed: May 17, 2004

Related U.S. Application Data
(60) Provisional application No. 60/531,601, filed on Dec. 23, 2003.

(51) Int. Cl. A61N 1/00

(52) U.S. Cl. 607/88

(57) ABSTRACT

Acoustic-optical therapy devices and methods for therapeutical purposes are disclosed in this invention. The devices provide a combination of ultrasound energy and optical pulsed energy exposed to human and animal body at frequencies of ultrasound and optical pulses within the range of 1 Hz to 1 GHz and at wavelengths from 0.2 micrometer to 20 micrometers. Both ultrasound energy and pulsed light radiation are delivered in effective combinations to maximize the therapy. Because of different interaction nature of ultrasound and light with body, the new invented devices will provide much more effective and specific body treatment.

### CROSS REFERENCE TO RELATED APPLICATIONS

This application is related to U.S. Provisional Patent Application No. 60/531,601, entitled “Acousto-Optic Therapeutical Devices and Methods” filed Dec. 23, 2003, which is herein incorporated by reference.

### STATEMENT REGARDING FEDERALLY SPONSORED RESEARCH

There is NO claim for federal support in research or development of this product.

### REFERENCES CITED

The following are patents found that may be associated with this information.

### U.S. Patent Documents

| | | |
|---|---|---|
| 5,080,101 | January 1992 | Dory |
| 5,080,102 | January 1992 | Dory |
| 5,471,988 | December 1995 | Fujio, et al. |
| 5,492,126 | February 1996 | Hennige, et al. |
| 5,666,954 | September 1997 | Chapelon, et al. |
| 5,697,897 | December 1997 | Buchholtz, et al. |
| 5,735,796 | April 1998 | Granz, et al. |
| 5,769,790 | January 1998 | Watkins, et al. |
| 5,788,636 | August 1998 | Curley |
| 5,873,828 | February 1999 | Fujio, et al. |
| 6,045,575 | April 2000 | Rosen, et al. |
| 6,290,713 | September 2001 | Russell, et al. |
| 6,443,978 | September 2002 | Zharov |
| 6,596,016 | July 2003 | Verman, et al. |

### OTHER REFERENCES

Lakowicz J. R. Time-Resolved Microscopy, Laser for Biotechnology, Laser Focus, (1992)
Rina Das, Apsara Dhokalia, Ellen V. Buchmann, Mary P. Kane, Margaret T. T. Wong-Riley, Rasha Hammamieh, Harry T. Whelan and Marti Jett LIGHT-EMITTING DIODE (LED) IRRADIATION ENHANCES THE WOUND HEALING PROCESS BY ALTERING GENE EXPRESSION PATTERNS, http://www.asc2002.com/Abstracts_only/d/DA-06.pdf (2002)
Smetana Z, Malik Z, Orenstein A, Mendelson E, Ben-Hur E., Treatment of viral infections with 5-aminolevulinic acid and light, Lasers Surg Med. 21(4):351-358 (1997)
Kuzdzal A. and Walaszek R., The use of visible incoherent polarised light in rehabilitation, Fizjoterapia, (10),3-4, 07 (2002)
Loo, C., Hirsch, L, Barton, J., Halas, N., West, J., and Drezek, R. “Nanoshell-Enabled Photonics-Based Cancer Imaging and Therapy.” Technology in Cancer Research and Treatment. 3(1): 33-40 (2004)
G. ter Haar, “Ultrasound Focal Beam Surgery,” Ultrasound in Medicine and Biology, Vol. 21, No. 9, pp. 1089-1100, (1995)
N. T. Sanghvi and R. H. Hawes, “High-intensity Focused Ultrasound,” Experimental and Investigational Endoscopy, Vol. 4, No. 2, pp. 383-395, (1994)

### FIELD OF THE INVENTION

This invention relates to body therapy methods and devices containing ultrasound and light sources of irradiation.

### BACKGROUND ART

Joint inflammation and edema to surrounding tissue are due to many medical conditions, blunt force trauma, physical exercise and stress to muscles, joints and tendons. To reduce such pain and suffering, which are associated with these conditions, common methods have been introduced to the public. This range from the use of external chemicals and ointments to sophisticated physical therapy applied to the area of inflammation in question. The principle behind this action is to stimulate blood flow and circulation to the effected area. Recently, light technology and photosensitizing agents have been used to reduce edema to surrounding tissues during pre and post surgical procedures to sensitive areas such as in the field of Ophthalmology.

Previous discovery and research in the field of Light Therapy (Phototherapy) has been well documented over the past 40 years. Worldwide independent research has demonstrated and proven that this type of technology will deliver powerful therapeutic benefits as it is applied to living tissues and organisms. Twenty-four (24) different positive changes, at a cellular level, have been documented using the Visible Light and Near Infrared Spectra Tests have proven that red light, at a wavelength of 660 nm, penetrates tissue to a depth of 8 to 10 mm (Lakowicz J. R., Time-Resolved Microscopy, Laser for Biotechnology, Laser Focus, 1992). Due to this penetration of light, it has been found to be beneficial in treating problems associated with injuries close the dermal layer surface and have been identified for the treatment of wounds, cuts, scars, nerve, acupuncture points and triggers (Rina Das, Apsara Dhokalia, Ellen V. Buchmann, Mary P. Kane, Margaret T. T. Wong-Riley, Rasha Hammamieh, Harry T. Whelan and Marti Jett LIGHT-EMITTING DIODE (LED) IRRADIATION ENHANCES THE WOUND HEALING PROCESS BY ALTERING GENE EXPRESSION PATTERNS, http://www.asc2002.com/Abstracts_only/d/DA-06.pdf). It has been found to be very successful in the treatment of infections (Smetana Z, Malik Z, Orenstein A, Mendelson E, Ben-Hur E., Treatment of viral infections with 5-aminolevulinic acid and light, Lasers Surg Med. 1997;21(4):351-358).

One of the amazing characteristics of the diverse tissue and cell types found in the human body is the fact that all have their own unique light absorption characteristics. Ultraviolet through visible light is absorbed in tissue by amino acids, proteins, NADH family compounds, collagen, Riboflavins. Light at this part of spectrum cause biochemical changes in tissue, which can leads to therapeutic biostimulation or to damaging tissue (Kuzdzal A. and Walaszek R., The use of visible incoherent polarised light in rehabilitation, Fizjoterapia, (10),3-4, 07). In the red and Near Infrared part of spectrum hemoglobin is directly associated with absorption of light at wavelengths in the 630-850 nm range, where Melanin (cells associated with pigmentation of skin) has a light absorption peak at 830 nm (Near Infrared). Although both red and infrared wavelengths penetrate to different depths and affect tissues differently, there overall therapeutic effects of light are quite similar. The both red and infrared light mainly generates heat in tissue. Heat has long been known to have many beneficial and necessary effects in the rehabilitation process. Heat increases the extensibility of collagen tissue, decreases joint stiffness, produces pain relief, relieves muscle spasm, increases blood flow, increases local metabolism, increases nerve conduction velocities, and assists in the resolution of inflammatory infiltrates, edema, and exudates. Heat has also been used as part of cancer therapy (Loo, C., Hirsch, L, Barton, J., Halas, N., West, J., and Drezek, R. “Nanoshell-Enabled Photonics-Based Cancer Imaging and Therapy.” Technology in Cancer Research and Treatment. 3(1): 33-40 (2004)).

Several inventions describe therapeutical devices and methods for light body treatment. U.S. Pat. No. 6,045,575 by Rosen et al. (2000) discloses a therapeutic method and a internally illuminated garment for the management of disorders treatable by phototherapy. The method according to the invention vests the infant in the garment, and energizes the light sources by coupling a battery to the light sources, or fueling and starting the fuel cell.

U.S. Pat. No. 6,290,713 by Russell (2001) discloses flexible illuminators for phototherapy. The flexible illuminators may be wrapped around an infant or a limb of an adult, or may be provided in larger configurations, such as a mat.

U.S. Pat. No. 6,443,978 by Zharov (2002) discloses a device for the physiotherapeutic irradiation of spatially extensive pathologies by light with the help of a matrix of the sources of optical radiation such as lasers or light diodes placed on the surface of a substrate whose shape is adequate to the shape of the zone of pathology.

U.S. Pat. No. 6,596,016 by Verman (2003) discloses a phototherapy garment contains a flexible backing material, a transparent liner, and a flexible printed circuit sheet containing surface-mounted light-emitting diodes (LEDs) positioned between the backing material and the liner. An infant is placed inside the garment so that the LEDs illuminate a large portion of the infant's skin for phototherapy.

After hot packs, ultrasound is probably the most frequently used physical agent in treating musculoskeletal pain and soft tissue injuries. Millions of ultrasound treatments are performed each year in the United States and Canada. Ultrasound produces the desirable therapeutic effects of any deep-heat modality. The effect of ultrasound that may be the most distinguishable, however, is its ability to selectively increase the temperature in local, well-circumscribed areas.

Ultrasound is a form of acoustic vibration occurring at frequencies too high to be perceived by the human ear. Thus, frequencies under 17,000 Hz are usually called sound, whereas those above this level are designated ultrasound. With the exception of the differences in frequencies, the physics of ultrasound is in no way different from that of audible sound. Ultrasonic frequencies typically used for therapeutic purposes range between 0.8 and 3 MHz.

The temperature distribution produced by ultrasound is unique among deep-heating modalities. Ultrasound causes comparatively little temperature elevation in the superficial tissues, but has a depth of penetration in the musculature and other soft tissues. In normal biological applications, for example, about 50% of the ultrasound energy is transmitted to a depth of 5 cm or greater, and this depth of penetration can be effectively employed in reaching deep tissues, such as joint capsules and deep muscles. For this reason, ultrasound is generally the treatment of choice when it is desirable to provide deep heat. When ultrasound energy is absorbed by tissue, it becomes thermal energy, raising the temperature of the tissue. To avoid thermal damage to tissue, the power level in diagnostic ultrasound imaging is kept very low. The typical ultrasound intensity (power per unit area) used in imaging is less than 0.1 watt per square centimeter. High intensity focused ultrasound, which can have an intensity above 1000 watts per square centimeter, can raise the tissue temperature at the region of the spatial focus to above 60-80 degrees Celsius in a few seconds and can cause tissue necrosis almost instantaneously.

High intensity ultrasound has been proposed to treat and destroy tissues in the liver (G. ter Haar, “Ultrasound Focal Beam Surgery,” Ultrasound in Medicine and Biology, Vol. 21, No. 9, pp. 1089-1100, 1995); in the prostate (N. T. Sanghvi and R. H. Hawes, “High-intensity Focused Ultrasound,” Experimental and Investigational Endoscopy, Vol. 4, No. 2, pp. 383-395, 1994); and in other organs. In U.S. Pat. Nos. 5,080,101, 5,080,102, 5,735,796, 5,769,790, and 5,788,636, for example, ultrasound imaging is combined with a high intensity ultrasound treatment to target the treatment region and to monitor the treatment process. In U.S. Pat. Nos. 5,471,988, 5,492,126, 5,666,954, 5,697,897, and 5,873,828, endoscopic ultrasound devices with both imaging and therapeutic capabilities are disclosed. These devices all have an elongated tube or shaft, so that they can be inserted in organ cavities (e.g., into the rectum) or into the abdominal cavity through a puncture hole in the abdominal wall to bring the ultrasound imaging and treatment sources closer to the disease sites. Some of them have flexible ends, which can be bent to fit the anatomy of a specific patient.

The therapeutic ultrasound beam can be focused inside tissue to a small spot of a few millimeters in size. At the focus, tissue temperature rapidly exceeds a level sufficient to cause tissue necrosis, thus achieving the desired therapeutic effect. Outside of the focus, ultrasound energy is less concentrated, tissue temperature rise remains below the necrosis level during the typically short exposure times employed. To treat a tissue volume larger than the focal spot, in the prior art, the ultrasound focus is deflected mechanically or electronically to scan, or incrementally expose, the target tissue volume. One disadvantage of the current high intensity ultrasound therapy is its inefficiency when treating large tumors or heating a large volume of tissue. Even though a three-second ultrasound pulse can increase the temperature of tissue at its focus dramatically, the ultrasound treatment must typically pause 40-60 seconds between two subsequent pulses to allow the intermediate tissue between the focus and the acoustic transducer to cool sufficiently to avoid thermally damaging the tissue. The volume of tissue necrosis for each treatment pulse is very small (.about.0.05 cm.sup.3). For example, to treat a volume of tissue within a 3 cm diameter sphere, it will take more than 4 hours, too long to be practical in most clinical situations. Many symptomatic uterine fibroids are larger than 2-3 cm in diameter, and multiple fibroids are also common. To be acceptable for clinicians and patients, the ultrasound treatment time must be significantly reduced.

### SUMMARY OF THE INVENTION

This invention relates to methods, designs and use of new acoustic-optical devices for therapeutical purposes. The device provides a combination of ultrasound energy and pulsed light energy exposed to human and animal body. Because of different nature of interaction of ultrasound and light with body, the new invented device will provide much more effective and specific body therapy.

The acoustic-optical device comprises an acoustic transducer and an optical source in the manner of combining irradiation of ultrasound and light on the targeted body. The ultrasound and pulsed light frequencies and wavelengths of light are design for most effective body therapy and for treatment of specific body concerns.

Another embodiment of the invention is to design the acoustic-optical device for specific therapeutical applications. Pluralities of designs are considered as embodiments in the present invention.

The invention also includes the use of the acoustic-optical device for reducing body inflammation, sinus symptoms, bacterial and viral infection, wound healing, nerve regeneration. These positive effects on body treatment are related to the use of ultrasound and pulsed light in the acoustic-optical device. Both ultrasound and light are transparent or semi-transparent to body and are biointeractive at specific frequencies and wavelengths.

### BRIEF DESCRIPTION OF THE DRAWINGS

FIG. 1. A flexible body wrap comprises of an ultrasound generator/source 3, optical source/LEDs 2 a flexible material substrate 1, and an electrical source 6.

FIG. 2. A flexible body wrap comprises of an ultrasound generator source 3, an optical source/laser 4, optical fibers/light guides 5, a flexible material substrate 1, and an electrical source 6. The ultrasound generator/source 3 is built into flexible substrate 1.

FIG. 3. A flexible body wrap comprises of an ultrasound generator source 3, an optical source/laser 4, optical fibers/light guide 5, a flexible material substrate 1, and an electrical source 6. The ultrasound generator/source 3 and optical source/laser 4 are coupled with optical fibers/light guides 5. Optical fibers/light guides 5 deliver the ultrasound and light to the flexible substrate 1.

FIG. 4. A dental insert comprises of an ultrasound generator source 3, optical source/LEDs 2 a rigid/flexible material substrate 1, and an electrical source 6. The ultrasound generator/source 3 and optical source/LEDs 2 are built into rigid or flexible substrate 1.

FIG. 5. A dental insert comprises of an ultrasound generator source 3, optical source/laser 4, a rigid/flexible material substrate 1, a rigid connector 7 within optical fibers/light guides 5 and an electrical source 6. The ultrasound generator source 3 and optical source/LEDs 2 are built into rigid/flexible substrate 1.

FIG. 6. An acoustic-optical device placed in a bathtub comprises of an ultrasound generator source 3, optical source/laser 4, a bathtub wall 9, optical fibers/light guides 5 and an electrical source 6. The ultrasound generator/source 3 and optical source/laser 4 are built into the bathtub wall 9.

FIG. 7. An acoustic-optical device placed in a shower cabin comprises of an ultrasound generator source 3, optical source/LEDs 2, a shower cabin wall 12, and an electrical source 6. The ultrasound generator/source 3 and optical source/laser 4 are built into the shower cabin wall 12.

FIG. 8. A hand-held acoustic-optical device comprises of an ultrasound generator source 3, optical source/LEDs 2, a handle 10, a substrate 11, and an electrical source 6. The ultrasound generator/source 3 and optical source/LEDs 2 are built into the hand-held acoustic-optical device substrate 11.

FIG. 9. An ear acoustic-optical therapy insert comprises of: an ultrasound generator/source 3, an optical source LEDs 2, a ear mount 12, a substrate 1, and an electrical source 6.

### DETAILED DESCIPTION OF THE INVENTION

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

The present invention provides an acoustic-optical therapy device and methods for therapeutical purposes. The device generates ultrasound and pulsed light which irradiate a targeted body region for purposes of a massage, tissue regeneration, nerve regeneration, hearing loss treatment, hair growth, skin treatment, removing tattoo, skin tanning, biostimulation, chemical decontamination, hygiene, drug delivery, treating bacterial infection, treating viral infection, treating sinus symptoms, treating pain, treating inflammation, wound healing, but not limited to them. Any suitable type of the device is included within the scope of the present invention, with all devices constructed from a light source and an ultrasound source described below.

The present invention enjoys many of its advantages due to its use of acoustic transducers and miniature light emitting diodes (LEDs)/laser semiconductors to deliver pulsed light directly to the targeted body. The acoustic transducers and LEDs/laser semiconductors are very small, very durable, and long-lasting. As a result, the devices are portable, lightweight, comfortable, easy to use, and relatively inexpensive. Acoustic transducers and LEDs/laser semiconductors deliver relatively high light intensity for their physical size and weight with relatively low power consumption.

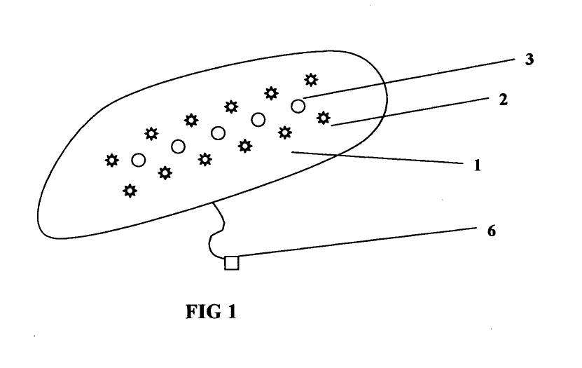

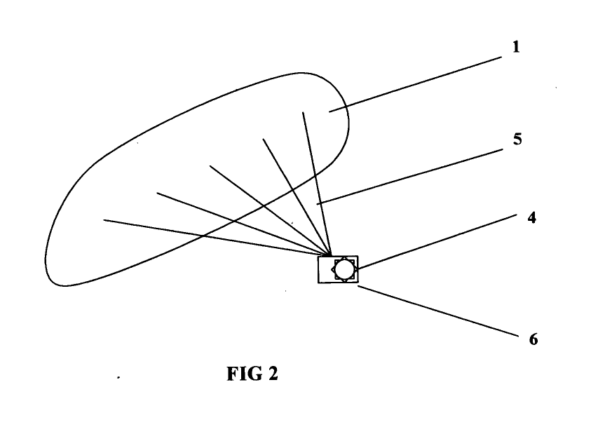

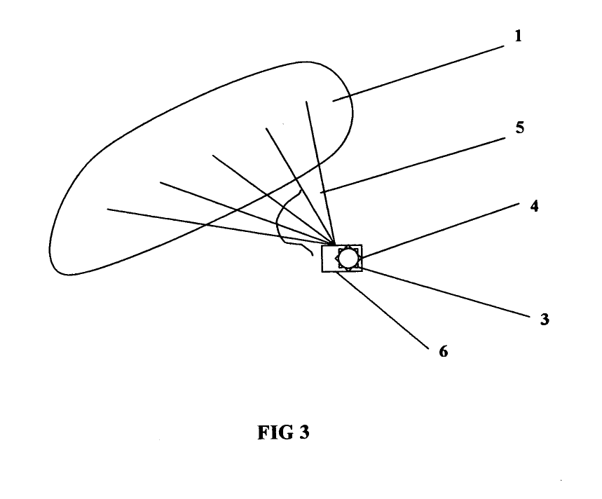

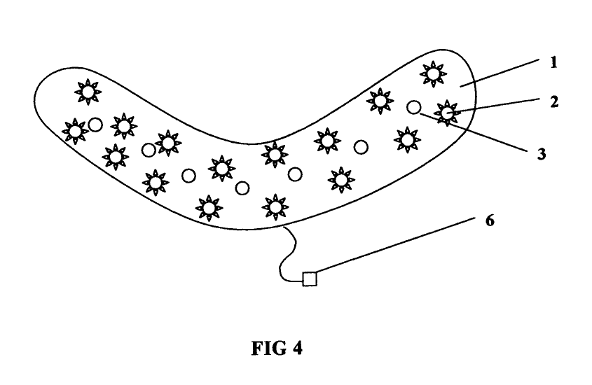

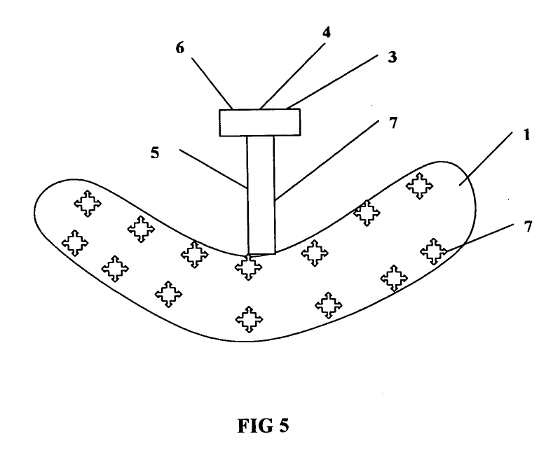

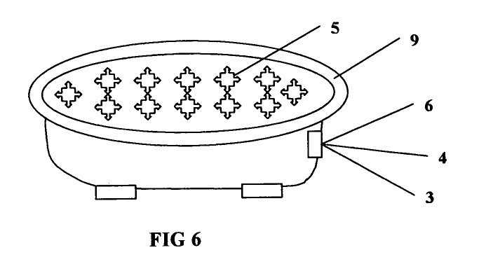

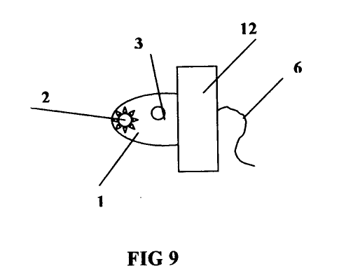

In particular, the present invention uses surface-mounted LEDs, also known as chip-type LEDs, as well common lens-type LEDs. Lens LEDs contain a relatively large bulbous lens used to focus the light at a particular angle, with two electrical leads extending from the bottom of the diode. They are thus relatively bulky and difficult to mount on a flexible surface, therefore lens type LEDs a very well suited for incorporating into the device with rigid surfaces like bathtub walls, shower cabin walls, sauna walls, but not limited to them (FIG. 6). In contrast, surface-mounted LEDs are very small and are mounted with their largest face contacting the surface and the leads extending sideways, so that they can easily be connected in series. Surface-mounted LEDs are therefore very well suited for incorporating into the device with flexible surfaces like flexible body wraps (FIG. 1, FIG. 2 and FIG. 3), body inserts (FIG. 4, FIG. 5 and FIG. 9), but not limited to them.

The present invention uses also semiconductor lasers which can be directly incorporated to the surface of the device or indirectly through optical fibers or light guides as is shown on the FIG. 1 and FIG. 2, respectively.

Regarding ultrasound sources, the present invention considers incorporating to the acoustic-optical device focused transducers and/or common unfocused transducers. Focused transducers can be used for direct irradiation of the targeted body region, therefore these transducers are incorporated into surface of the device or buried inside the device (FIG. 1, FIG. 4 and FIG. 9). Focused transducers can be also used in indirect irradiation of the targeted body region, by focusing ultrasound energy on optical fiber ends and transferring ultrasound energy through the fiber to the targeted body (FIG. 3, FIG. 5 and FIG. 6). Unfocused transducers are preferably placed on surface of the device that allows for direct contact of transducers with medium and/or with the targeted body region.

The presently preferred embodiments of the invention will be best understood by reference to the drawings, wherein like parts are designated by like numerals throughout. One presently preferred embodiment of the acoustic-optical therapy device of the present invention applied as a flexible body wrap is illustrated in FIG. 1. As shown, the flexible body wrap comprises of: an ultrasound generator/source 3, an optical source LEDs 2 an elastic material substrate 1, and an electrical source 6. The wrap can be used on any part of body for therapeutic purposes. This embodiment considers also use the semiconductor lasers 4 (FIG. 2, FIG. 3, FIG. 5) instead LEDs, or using both lasers and semiconductors. The lasers can be mounted into surface of the device similar way as LEDs (FIG. 1), or as is shown on FIG. 2, the laser 4 can be optically coupled to optical fibers/light guides 5. In this design the ultrasound generator/source can also be coupled with optical fibers/optical light guides 5 or be placed directly on the flexible substrate 1 of the device. The flexible body wraps presented on FIG. 1, FIG. 2 and FIG. 3 are examples of possible designs used for covering any body part and are considered as embodiments of the present invention. Another preferred embodiment of the acoustic-optical therapy device of the present invention applied as a dental insert is illustrated in FIG. 4 and FIG. 5. As shown, the dental insert comprises of: an ultrasound generator/source 3, an optical source LEDs 2, a plastic type rigid material substrate 1, and an electrical source 6. The dental insert designs showed on FIG. 4 and FIG. 5 are only examples of many other designs considered as embodiment of the present invention.

Another preferred embodiment of the acoustic-optical therapy device of the present invention is a bathtub acoustic-optical therapy device which is illustrated in FIG. 6. As shown, the bathtub device comprises of: an ultrasound generator/source 3, an optical source LEDs 2, a bathtub wall substrate 9, and an electrical source 6. The bathtub device design showed on FIG. 6 is an example of many other designs and applications of the acoustic-optical therapy device considered as embodiments of the present invention, like a body part bathtub, whirlpool bathtub, Jacuzzi bathtub, shower cabin, sauna cabin, bathroom, chair, pillow, comforter, blanket, mattress, bed, sofa cushions, armchair, back rest, seat cushion, lamp, car seat, personal clothing, but not limited to them.

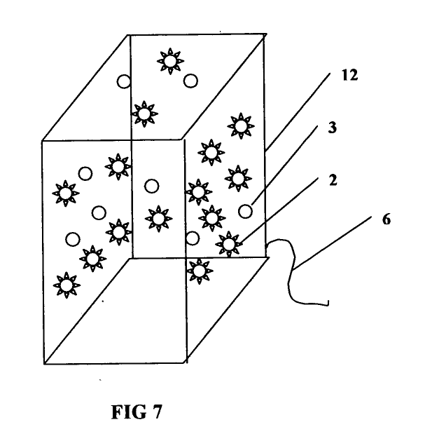

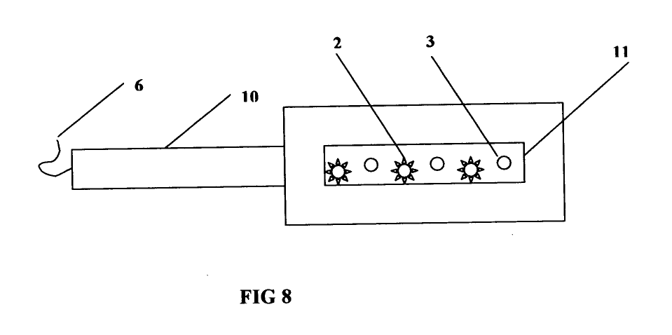

One presently preferred embodiment of the acoustic-optical therapy device of the present invention applied to a shower cabin is illustrated in FIG. 7. As shown, the shower cabin comprises of: an ultrasound generator/source 3, an optical source LEDs 2, a rigid material substrate 12, and an electrical source 6. The shower cabin design showed on FIG. 7 is an example of many other designs considered as embodiments of the present invention. Another presently preferred embodiment of the acoustic-optical therapy device of the present invention is a hand-held acoustic-optical therapy device which is illustrated in FIG. 8. As shown, the hand-held device comprises of: an ultrasound generator/source 3, an optical source LEDs 2, a handle 10, a substrate 11, and an electrical source 6. The hand-held device design showed on FIG. 8 is an example of many other designs and applications of the hand-held acoustic-optical therapy device considered as embodiments of the present invention.

Another presently preferred embodiment of the acoustic-optical therapy device of the present invention is an ear acoustic-optical therapy insert illustrated in FIG. 9. As shown, the ear acoustic-optical therapy insert comprises of: an ultrasound generator/source 3, an optical source LEDs 2, a ear mount 12, a substrate 1, and an electrical source 6. The ear acoustic-optical therapy insert design showed on FIG. 9 is an example of many other designs and applications of the ear acoustic-optical therapy insert considered as embodiments of the present invention.

It will be appreciated by a person of average skill in the art that the present invention uses for therapeutical purpose a combination of ultrasound and pulsed light at frequencies from 1 Hz to 1 GHz and wavelengths from 0.2 micrometer to 20 micrometers. The ultrasound at those frequencies may deposit heat and destroy bacteria in the targeted body. The pulsed light at those frequencies may affect body similar way as the ultrasound and in addition it may stimulate wavelength dependent biochemical processes in the targeted body. These biochemical processes are enhanced in the presence of ultrasound mainly due to raised temperature of targeted body. In consequence, new or more intense biochemical processes will be induced in the targeted body under irradiation of ultrasound and pulsed light, e.g. skin tanning can be performed with light at longer wavelengths than carcinogenic ultraviolet light, hearing can be improved by nerves and hair cells regeneration or by reducing ear infection, skin tattoo can be removed more efficient due to enhanced ink photobleaching and cell membranes burst, hair growth can be increased by better nutrients delivery, muscles and joints pain can be reduced by increased blood flow in tissue and by eliminating bacterial infection. These are only few application examples of acoustic-optical therapy devices and methods considered as embodiments of the present invention.

1. An acoustic-optical therapy device and method comprising of:
a. a targeted body region for therapeutical purposes;
b. an acoustic transducer for generating ultrasound used in irradiating said targeted body region for therapeutical purposes;
c. an optical source for generating pulsed light used in irradiating said targeted body region for therapeutical purposes;
d. an acoustic transducer and an optical source arranged in such a manner that ultrasound and pulsed light are irradiating said targeted body region;
e. a medium through which said targeted body region is irradiated by ultrasound and pulsed light;
f. an electrical source for supplying electrical energy to said acoustic transducer and said optical source.

2. An acoustic-optical therapy device as claimed in claim 1 wherein said optical source is an LED, laser, semiconductor laser, lamp, fluorescence, electroluminescence.

3. An acoustic-optical therapy device as claimed in claim 1 and claim 2, wherein said optical source is a pulsed light source at frequencies within the range of 1 Hz to 1000 MHz.

4. The method of claim 3, wherein said optical source has a pulse duration of less than 500 ms.

5. An acoustic-optical therapy device as claimed in claim 1 and claim 2, wherein said optical source has intensity within the range of 0.000005-50 W/cm2.

6. An acoustic-optical therapy device as claimed in claim 1 and claim 3, wherein: said optical source in said acoustic-optical therapy device is a single frequency pulsed light source, multiple frequencies pulsed light source.

7. An acoustic-optical therapy device as claimed in claim 1 and claim 2, wherein said optical source generates wavelengths within the range of 0.2 micrometer to 20 micrometers.

8. An acoustic-optical therapy device as claimed in claim 1 and claim 2, wherein said optical source emits monochromatic light, polychromatic light.

9. An acoustic-optical therapy device as claimed in claim 1 and claim 2, wherein said optical source emits polarized light, unpolarized light.

10. An acoustic-optical therapy device as claimed in claim 1, wherein said acoustic transducer generates ultrasound at frequencies within the range of 1 Hz to 1000 MHz.

11. An acoustic-optical therapy device as claimed in claim 1 and claim 10, wherein said acoustic transducer generates ultrasound at intensities within the range of 0.000005-50 W/cm2.

12. An acoustic-optical therapy device as claimed in claim 1 and claim 10, wherein said acoustic transducer in said acoustic-optical therapy device is using a single ultrasound frequency, multiple ultrasound frequencies.

13. An acoustic-optical therapy device as claimed in claim 1, wherein said medium is air, water, spray, optical fiber, light guide, organic material, inorganic material, polymer, gel, ointment, mineral oil, paraffin, wax, organic oil, targeted body.

14. An acoustic-optical therapy device as claimed in claim 1, wherein said electrical source for supplying electrical energy to said acoustic transducer and said optical source is a battery, AC current source, DC current source, solar battery.

15. An acoustic-optical therapy device and method as claimed in claim 1, wherein said targeted body is an external body region, internal body region, external and internal body region.

16. An acoustic-optical therapy device and method as claimed in claim 1, wherein said acoustic-optical therapy device is applied in said targeted body for a massage, tissue regeneration, nerve regeneration, hearing loss treatment, hair growth, skin treatment, tattoo removal, skin tanning, hair removal, chemical decontamination, hygiene, drug delivery, treating bacterial infection, treating viral infection, treating sinus symptoms, treating pain, treating inflammation, wound healing.

17. An acoustic-optical therapy device as claimed in claim 1 and claim 16, wherein said acoustic-optical therapy device is a hand-held portable device.

18. An acoustic-optical therapy device as claimed in claim 1 and claim 17, wherein said acoustic-optical therapy device is a toothbrush.

19. An acoustic-optical therapy device as claimed in claim 1 and claim 17, wherein said acoustic-optical therapy device is a hairbrush.

20. An acoustic-optical therapy device as claimed in claim 1 and claim 16, wherein said acoustic-optical therapy device is a body insert.

21. An acoustic-optical therapy device as claimed in claim 1 and claim 16, wherein said acoustic-optical therapy device is a dental insert.

22. An acoustic-optical therapy device as claimed in claim 1 and claim 16, wherein said acoustic-optical therapy device is an ear insert.

23. An acoustic-optical therapy device as claimed in claim 1 and claim 16, wherein said acoustic-optical therapy device is a flexible wrap covering said targeted body region.

24. An acoustic-optical therapy device as claimed in claim 1 and claim 16, wherein said acoustic-optical therapy device is used in a bathtub, body part bathtub, whirlpool bathtub, Jacuzzi bathtub, shower cabin, sauna cabin, bathroom, chair, pillow, comforter, blanket, mattress, bed, sofa cushions, armchair, back rest, seat cushion, lamp, car seat, personal clothing.

25. An acoustic-optical therapy device as claimed in claim 1 and claim 16, wherein said acoustic-optical therapy device and methods are used in combination with magnetic therapy, electrophysiology therapy, mechanical massage therapy, heat therapy.

26. An acoustic-optical therapy device and method as claimed in claim 1 wherein said acoustic-optical therapy device is applied for biological or chemical decontamination of said targeted body, food, material surface, clothing, air, water.
